(19) United States
(12) Patent Application Publication
(10) Pub. No.: US 2025/0169766 A1
(43) Pub. Date: May 29, 2025

(54) SYSTEMS AND METHODS FOR IMPROVING ECG SIGNAL QUALITY WITH INDUCED ELECTRICAL SIGNALS

(71) Applicant: KONINKLIJKE PHILIPS N.V., Eindhoven (NL)

(72) Inventors: Eduard Gerard Marie Pelssers, Eindhoven (NL); Gabriele Papini, Eindhoven (NL); Hugo Veenstra, Eindhoven (NL); Anton Petrus Gerardus Emanuel Janssen, Eindhoven (NL); Jens Muehlsteff, Eindhoven (NL)

(21) Appl. No.: 18/953,356

(22) Filed: Nov. 20, 2024

Related U.S. Application Data
(60) Provisional application No. 63/603,287, filed on Nov. 28, 2023.

(51) Int. Cl. A61B 5/00; A61B 5/308; A61B 5/318

(52) U.S. Cl. CPC A61B 5/7221; A61B 5/308; A61B 5/318

(57) ABSTRACT

Systems and method for determining a fidelity metric of an ECG signal are provided. The system of method including a signal generator configured to induce an ECG test signal into a tissue of a patient via a first plurality of electrodes. A signal processor receives an ECG signal including a test signal portion and a heart signal portion. The signal processor detects one or more disturbance in the ECG signal based on a comparison between the test signal portion and the ECG test signal. The signal processor then determines a disturbance level for the test signal portion based on the one or more detected disturbances and a fidelity metric for the heart signal portion based on the determined disturbance level of the test signal portion.

### FIELD OF THE DISCLOSURE

The present disclosure generally relates to electrocardiography measurements. More specifically, the present disclosure is directed to methods and systems for improving the reliability of such measurements by determining a fidelity metric for electrocardiogram signals based on induced ECG test signals.

### BACKGROUND

An electrocardiogram (ECG or EKG) is a non-invasive medical test that measures and records the electrical activity of the heart over a particular period of time, usually a few seconds to a few minutes. It provides valuable information about the heart's rhythm, rate, and overall function. The ECG recording takes the form of a graph of voltage versus time, showing the heart's rhythm and detecting abnormalities in its functioning via analysis of a series of waveforms, which are the essential components of the ECG and are used to diagnose various heart diseases by medical professionals. ECGs are widely employed for monitoring known heart conditions, diagnostics of specific cardiac symptoms, preoperative assessment of cardiac patients, or routine cardiac check-ups of individuals with a family history of heart diseases or other risk factors.

However, with the exceptions of certain populations and occupations at higher risk of cardiac conditions, such as professional athletes, pilots, astronauts and the like, ECG is not generally used as a means for routine screening of healthy persons, or persons at low risk, at least in part due to the potential for false positives often stemming from a high rate of signal disturbances.

Accordingly, there still exists a need in the art for systems and methods capable of accurately detecting and isolating signal disturbances in ECG measurements. Once such signal disturbances are managed, e.g. by filtering, the possible applications of ECG could be expanded from the periodic monitoring of only individuals with known heart health problems, to continuous screening and preventative monitoring of healthy individuals as well. This is especially true in view of the advent of wearable devices, such as smart watches, capable of providing ECG measurements.

### SUMMARY OF THE DISCLOSURE

The present disclosure is generally directed to systems and methods for determining a fidelity metric for ECG signals based on signal disturbances detected in an ECG test signal. This determination is achieved partially through the realization that artificial electrical signals can be induced into tissue of a patient to create a second “artificial heart” for the ECG to detect. However, unlike the patient's heart, the signals generated by this “artificial heart” are known prior to the ECG measurement. Accordingly, the detected ECG output may be compared to the known test signal input to detect and characterize any disturbances in the ECG measurement. This disclosure is also based, at least partially, on the further insight that signal disturbances affecting the test signal are also likely to impact the ECG heart signal measurements. Accordingly, detected disturbances in the test signal may be used as a proxy for determining a fidelity metric for the ECG heart signal measurements as well.

Generally, one aspect of this disclosure relates to an ECG system. The ECG system includes a signal generator configured to induce an ECG test signal into a tissue of a patient via a first plurality of electrodes. The system further includes a second plurality of electrodes configured to attach to skin of the patient and to detect an ECG heart signal, and further configured to detect the ECG test signal. The system further includes a signal processor configured to: i) receive an ECG signal comprising a heart signal portion, based on the detected ECG heart signal, and a test signal portion, based on the detected ECG test signal, ii) detect one or more disturbances in the test signal portion based on a comparison between the test signal portion and the induced ECG test signal, iii) determine a disturbance level for the test signal portion based on the one or more disturbances, and iv) determine a fidelity metric for the heart signal portion based on the disturbance level.

In some embodiments, the signal processor is further configured to filter out at least a portion of the ECG signal upon detection of the one or more disturbances.

According to some examples, determining the fidelity metric further includes comparing the disturbance level to a disturbance threshold, and filtering out at least a part of the ECG signal when the disturbance level meets or exceeds the disturbance threshold.

According to some examples, the signal processor is further configured to selectively control the signal generator so that the ECG test signal shares one or more spectral characteristics with the ECG heart signal.

In some embodiments, the ECG test signal includes a rectangular pulse wave, and the signal processor is further configured to determine one or more frequencies associated with the one or more disturbances.

In other embodiments, the ECG test signal includes a sine wave, and the signal processor is further configured to selectively control the signal generator to continuously increase the frequency of the sine wave, and the signal processor is further configured to determine one or more frequencies associated with the one or more disturbances based, at least in part, on the changing frequency of the sine wave.

According to some examples, the first plurality of electrodes and the signal processor are integrated into a wearable device.

In some embodiments, the signal processor is further configured to determine a first beat portion and a second beat portion of the ECG heart signal, based at least in part on the heart signal portion, and selectively control a timing of the signal generator to induce the ECG test signal between the first beat portion and the second beat portion of the ECG heart signal.

Another aspect of the present disclosure generally relates to a method for determining a fidelity metric of an ECG signal. The method includes inducing an ECG test signal into a tissue of a patient via a first plurality of electrodes. The method further includes receiving, an ECG signal including a heart signal portion and a test signal portion. The heart signal portion being based on an ECG heart signal detected by a second plurality of electrodes and the test signal portion being based on an ECG test signal also detected by the second plurality of electrodes. The method further includes detecting one or more disturbances in the test signal portion based on a comparison between the test signal portion and the induced ECG test signals. The method further includes determining a disturbance level for the test signal portion based on the one or more disturbances. The method further includes determining a fidelity metric for the heart signal portion based on the disturbance level.

In some embodiments, the method further includes: i) receiving a first ECG signal at a first time, ii) determining a patient heart rate based, at least partially, on the heart signal portion of the first ECG signal, iii) receiving a second ECG signal at a second time, iv) determining a change in the patient heart rate based, at least partially, on the heart signal portion of the second ECG signal, and v) determining a change in patient glucose level based, at least partially, on the determined change in patient heart rate.

According to some examples, the method further includes: i) determining a fidelity metric for each lead within the second plurality of electrodes, ii) comparing each of the fidelity metrics to a fidelity threshold, and iii) selectively recording the ECG signal detected by a lead within the second plurality of leads only when the fidelity metric corresponding to the lead meets or exceeds the fidelity threshold.

According to some examples, the method further includes filtering out at least a part of the ECG signal upon detection of the one or more disturbances.

According to some examples, the method further includes comparing the disturbance level to a disturbance threshold, and filtering out at least a part of the ECG signal when the disturbance level exceeds the disturbance threshold.

According to some examples, the method further includes selectively controlling a signal generator so that the ECG test signal shares one or more spectral characteristics with the ECG heart signal.

According to some examples, the method further includes determining a first beat portion and a second beat portion of the ECG heart signal, based at least in part on the heart signal portion, and selectively controlling a timing of a signal generator to induce the ECG test signal between the first beat portion and the second beat portion of the ECG heart signal.

In various implementations, a processor or controller can be associated with one or more storage media (generically referred to herein as “memory,” e.g., volatile and non-volatile computer memory such as ROM, RAM, PROM, EPROM, and EEPROM, floppy disks, compact disks, optical disks, magnetic tape, Flash, OTP-ROM, SSD, HDD, etc.). In some implementations, the storage media can be encoded with one or more programs that, when executed on one or more processors and/or controllers, perform at least some of the functions discussed herein. Various storage media can be fixed within a processor or controller or can be transportable, such that the one or more programs stored thereon can be loaded into a processor or controller so as to implement various aspects as discussed herein. The terms “program” or “computer program” are used herein in a generic sense to refer to any type of computer code (e.g., software, firmware, or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments and examples will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is an illustration of an ECG system according to some aspects of the present disclosure;

FIG. 2 is an illustration of an ECG heart signal according to some aspects of the present disclosure;

FIG. 3 is an illustration of an ECG signal including both a heart signal portion and a test signal portion according to some aspects of the present disclosure;

FIGS. 4A and 4B are illustrations of an ECG test signal being induced between two ECG heart signals according to some aspects of the present disclosure;

FIG. 5 is an illustration of a rectangular pulse wave and a continuously increasing sine wave according to some aspects of the pending disclosure;

FIG. 6 is flow chart of a method for determining a fidelity metric of an ECG signal according to some aspects of the present disclosure;

FIG. 7 is a diagram of a mobile ECG system according to some aspects of the present disclosure;

FIG. 8 is a flow chart illustrating a method for determining a change in patient glucose level according to some aspects of the pending disclosure; and

FIG. 9 is a flow chart illustrating a method for selectively recording ECG signals based on a comparison between different electrode leads according to some aspects of the pending disclosure.

### DETAILED DESCRIPTION OF EMBODIMENTS

The present disclosure is generally directed to systems and methods for determining a fidelity metric for ECG signals based on signal disturbances detected in an ECG test signal. Artificial electrical signals of known intensity, shape, and/or frequency are induced, via an electrode set, into the human body and are subsequently received by ECG-electrodes. In the ECG recordings, disturbances are detected, a fidelity metric is determined, and deviations from the originally known test signal are used to continuously correct and/or classify ECG signals with respect to the fidelity metric.

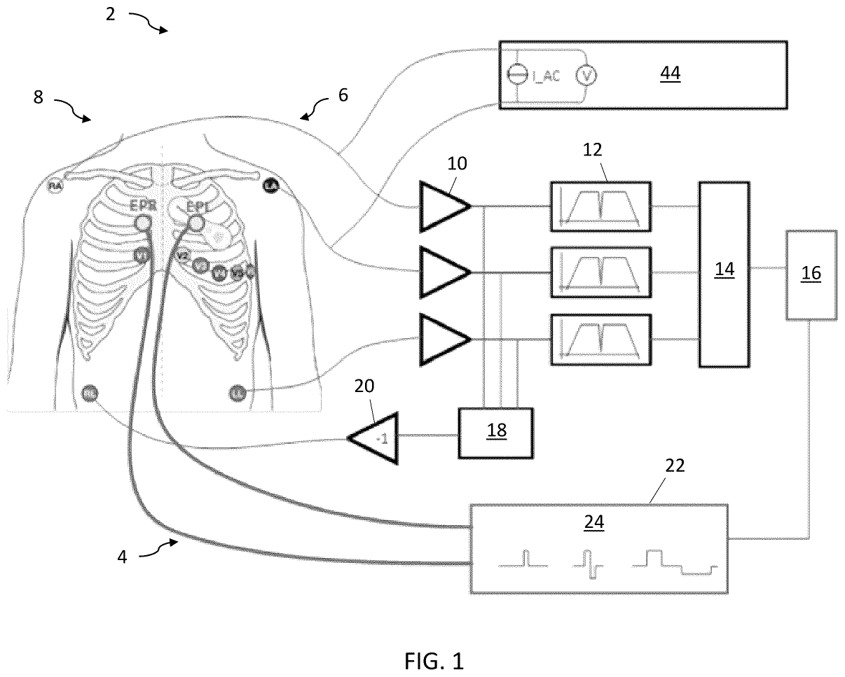

FIG. 1 illustrates an ECG system 2 according to some aspects of the present disclosure. A first plurality of electrodes 4 and a second plurality of electrodes 6 are shown as conductive pads attached to the body of patient 8. A conventional ECG measurement is performed using the 10electrodes of the second plurality of electrodes 6. As shown in FIG. 1, these 10 electrodes include electrodes V1-V6 placed along the torso of patient 8. Additional electrodes RA, LA, and LL are respectively placed at points along the right arm, left arm, and left leg of patient 8. ECG system 2 measures signals produced by the heart of patient 8 by detecting a voltage between electrodes. Accordingly, an electrode lead is defined relative to two electrodes. For example, in ECG system 2, lead I is between RA and LA, lead II is between RA and LL, and Lead III is between LA and LL to define Einthoven's triangle, as apparent to those of ordinary skill in the art.

Generally, the detected voltages will be amplified by one or more differential input amplifiers 10 and filtered by one or more filters 12 in preparation for processing by signal processor 16. The ECG system 2 of FIG. 1 illustrates filters 12 as notch filters. This filtering arrangement is useful for when signal noise is associated with a previously known frequency range. Additionally or alternatively, filters 12 could be implemented as one or more high pass filters, low pass filters, and/or bandpass filters. The voltage signals then pass through analog-to-digital converter 14 and undergo processing via signal processor 16.

Ideally the differential input amplifiers 10 would fully reject any common mode noise that is present on the cables, second plurality of electrodes 6, and body of patient 8. But mismatches in the electrode/body interface and finite capabilities of real-world amplifiers makes the final practical implementation less ideal. A known means for reducing common mode interference is by actively driving the body of patient 8. This is achieved by including an additional RLD electrode in the second plurality of electrodes 6. The RLD electrode is typically placed on the right leg as this point is furthest away from the heart. Under normal conditions, when all the electrodes appear to be attached properly, the RLD circuit 20 sets the body potential, but the RLD electrode can also be used to reject common mode noise. Sum module 18 is also known as the Wilson's Central Terminal to those of ordinary skill in the art. Sum module 18 serves as a common virtual electrode and is produced by averaging the measurements from the electrodes RA, LA, and LL to give an average potential of the body. This average potential is then used to derive the signal actively driven on the RLD electrode. This will suppress the common mode noise as picked up by the sensing electrodes. This is helpful, for example, for suppressing the known electromagnetic interference of 50 Hz in Europe or 60 Hz in the USA originating from other electrical devices within the room.

FIG. 1 further illustrates an additional component of signal generator 22, not included in conventional ECG systems. Signal generator 22 is configured to induce one or more ECG test signals 24 into a tissue of patient 8 via the first plurality of electrodes 4. Signal generator 22 can be implemented using a device embedded within patient 8, such as pacemaker. However, the signal generator 22 of FIG. 1 illustrates less invasive techniques where the signal generator 22 is implemented with the first plurality of electrodes 4 being driven by signal processor 16. Signal generator 22 can be positioned within a patient monitor, a wearable patch, or in a wearable consumer device such as a smart watch. The first plurality of electrodes 4 is shown as an additional pair to the second plurality of electrodes 6, however signal generator 22 could also generate the one or more ECG test signals 24 using a pair of the second plurality of electrodes 6. Signal generator 22 serves as an “artificial heart” in the sense that the ECG test signals 24 are detected by the second plurality of electrodes 6 just as if they were signals produced by the heart of patient 8. For safety reasons, the induced ECG test signals 24 should be compliant with FDA guidance.

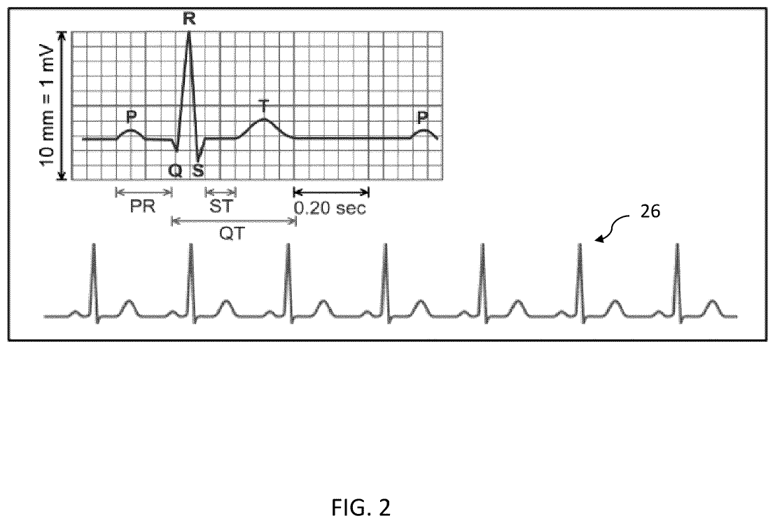

FIG. 2 is an illustration of an ECG heart signal 26 according to some aspects of the present disclosure. The ECG heart signal 26 is familiar to those of ordinary skill in the art and includes a P-wave portion P, a Q-wave portion Q, an R-wave portion R, a S-wave portion S, and a T-wave portion T. FIG. 2 further illustrates a PR interval from the beginning of P-wave portion P to the beginning of Q-wave portion Q, an ST segment from the end of S-wave S to the beginning of T-wave T, and a QT interval from the beginning of Q-wave Q to the end of T-wave T. FIG. 2 further illustrates a left axis of 1 mV for reference of the amplitude of the ECG heart signals 26, and a period of 20 seconds between the end of one ECG heart signal 26 and the beginning of another.

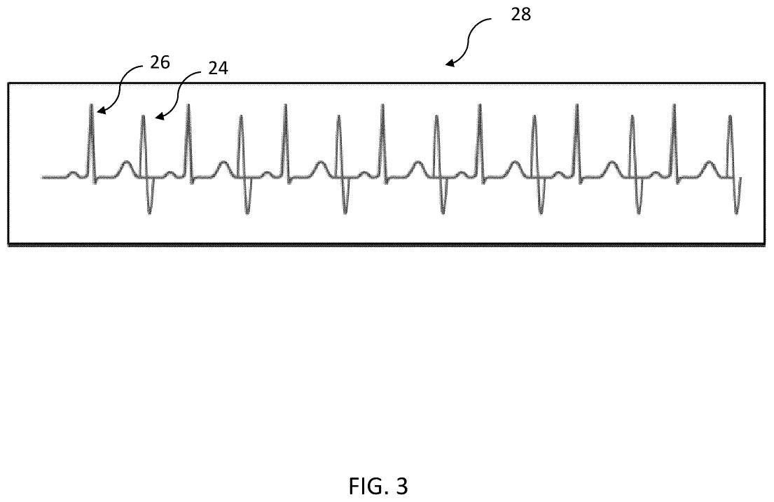

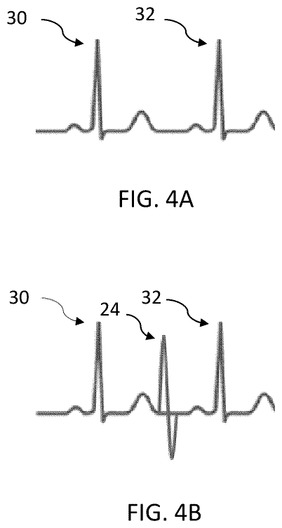

Turning to FIG. 3, signal processor 16 receives an ECG signal 28. ECG signal 28 includes a heart signal portion made up of the ECG heart signals 26 and a test signal portion made up of the ECG test signals 24. FIG. 3 shows the ECG test signals 24 induced by signal generator 22 to be between the individual ECG heart signals 26. With reference also to FIGS. 4A and 4B, the heart signal portion of ECG signal 28 is seen to include a plurality of ECG heart signals 26. Signal processor 16 is configured to determine a first beat portion 30 and second beat portion 32 based on the heart signal portion of ECG signal 28. The first beat portion 30 and the second beat portion 32 can be determined based on landmarks in the ECG signal 28. For example, detecting a T-wave T may indicate that first beat portion 30 is ending and a second beat portion 32 is going to begin. Signal processor 16 may then selectively the time control signal generator 22 to induce the ECG test signal 24 in between the first beat portion 30 and the second beat portion 32 of the ECG signal 28. This ensures that the ECG test signals 24 do not overlap with the ECG heart signals 26 which would introduce interference into both sets of signals. Signal processor 16 may further account for respiratory sinus arrythmia when determining a beat free region to induce ECG test signal 24. In this manner, signal processor 16 may account for the space between ECG heart signals 26 being smaller during periods when patient 8 is inhaling than during periods when patient 8 is exhaling. This respiratory sinus arrythmia may be determined according to systems and methods further elaborated upon herein.

ECG test signal 24 may take a variety of forms. In some examples, signal processor 16 is configured to selectively control signal generator 22 so that ECG test signal 24 shares one or more spectral characteristics with ECG heart signal 26. Such spectral characteristics can include the frequency, shape, and/or voltage of the ECG test signal 24. The ECG test signals 24 shown in FIG. 3 are selected to have a similar frequency and shape to the R-wave R of the ECG heart signals 26, with an approximately symmetrical negative portion to balance any stress experienced by patient 8 from the induced voltage of the ECG test signals 24. Since the artificial ECG test signals 24 are induced by the signal generator 22, the generated signals are exactly known in frequency, shape, and voltage. Moreover, any effect of the filters 12 as employed in a patient monitor, can be measured or simulated a-priori and the expected recorded signal is known a-priori. Accordingly, any further deviation can then be attributed to some form of signal disturbance. In some examples, the spectral characteristics of ECG test signal 24 are selected based on previous knowledge of the factors impacting signal fidelity. For example, the ECG test signals 24 may be configured to contain a frequency range that coincides with a known source of interference. In this way, ECG test signals 24 may be used to test how the ECG heart signals 26 will be distorted, or if they will in fact be distorted, at this range of frequencies.

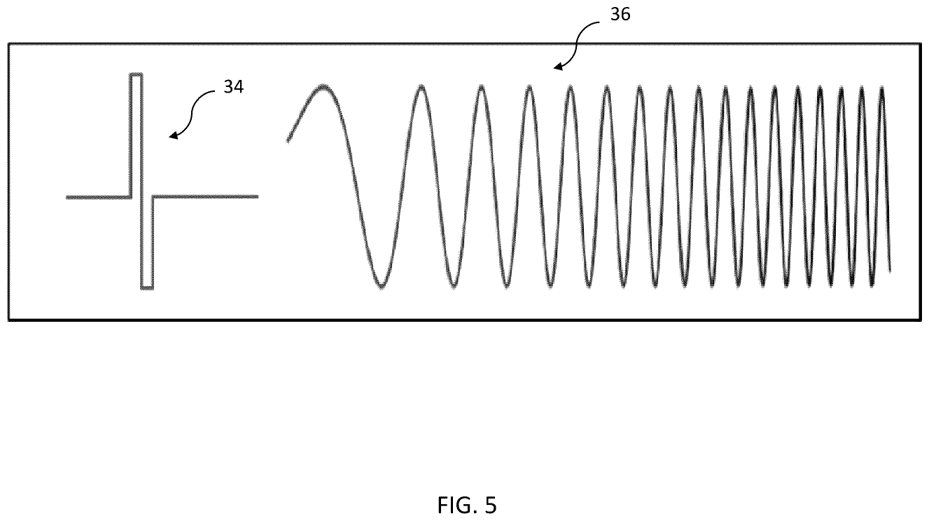

Some embodiments may include a spectral analysis of the ECG test signal 24 over many or all frequencies. Turning to FIG. 5, the ECG test signal 24 may include a rectangular pulse wave 34. When transformed into the frequency domain, a rectangular pulse is a Sinc function extending over all frequencies. Accordingly, signal processor 16 can determine a frequency associated with any disturbance in the generated rectangular pulse wave 34 by analyzing the signal in the frequency domain. Moreover, a filter, such as a band pass filter at the frequency of the detected disturbances, may then be applied to both the ECG test signals 24 and the ECG heart signals 26, to remove any such disturbances. A spectral analysis may similarly be performed where the ECG test signal 24 is a sine wave and signal processor 16 is configured to selectively control signal generator 22 to continuously increase the frequency of the sine wave. Such a continuously increasing sine wave 36 is shown in FIG. 5. When transformed into the frequency domain, the continuously increasing sine wave 36 is a series of impulses isolated at the various frequencies of the continuously increasing sine wave 36. Any distortion to this series of impulses is readily apparent when viewed in the frequency domain and may similarly be filtered out based on the frequency of the disturbances.

Notably, the ECG test signal 24 is not limited to any one signal configuration. For example, the systems and methods disclosed herein contemplate ECG signal 28 being tested with a series of different ECG test signals 24. Testing how the ECG system 2 responds to a variety of different ECG test signals 24, will generally lead to a more detailed and robust analysis of the fidelity of the ECG heart signals 26 and will allow any confounding factors to be more precisely isolated. Moreover, embodiments involving a spectral analysis over all frequency ranges would include separate parallel recordings by the second plurality of electrodes 6. This is necessary to avoid portions of the rectangular pulse wave 34 or continuously increasing sine wave 36 being subjected to filters 12.

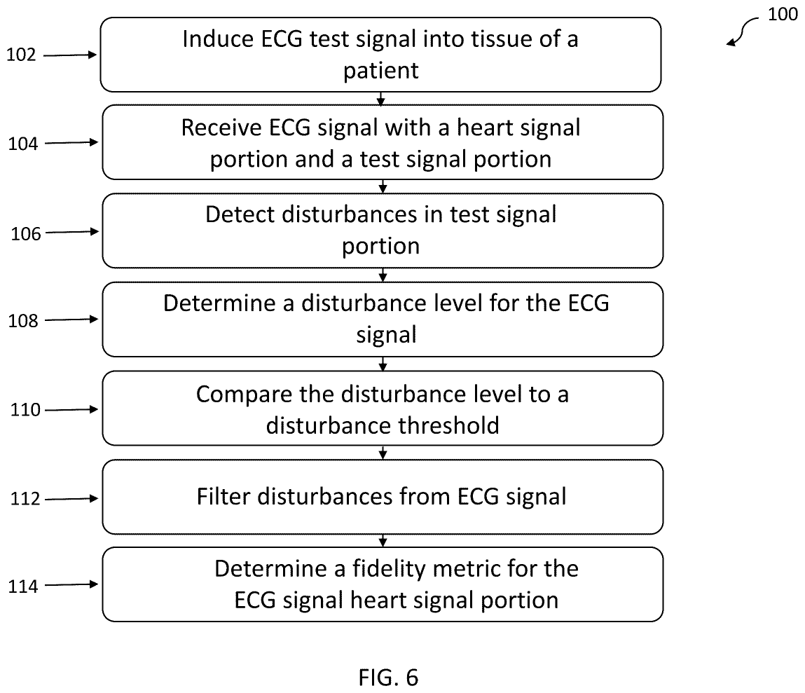

FIG. 6 illustrates a flow chart of a method 100 for determining a fidelity metric of an ECG signal. Step 102 of method 100 includes inducing an ECG test signal into a tissue of a patient. This step could be performed using previously discussed techniques. For example, by signal processor 16 controlling signal generator 22 to induce ECG test signal 24 into patient 8 via the first plurality of electrodes 4. Step 104 of method 100 includes receiving an ECG signal with a heart signal portion and a test signal portion. This step could be performed by signal processor 16 receiving ECG signal 28, shown in FIG. 3. The heart signal portion is based on an ECG heart signal detected by a second plurality of electrodes and the test signal portion is based on an ECG test signal also detected by the second plurality of electrodes. For example, the second plurality of electrodes 6 could detect both ECG heart signals 26 and ECG test signals 24 so that the ECG signal 28 containing both set of signals is transmitted to signal processor 16. Step 106 of method 100 includes detecting one or more disturbances in the test signal portion. There are a variety of possible sources for disturbances in an ECG measurement. Some examples of sources of disturbances include patient breathing, shifts in the patient's body and/or limb position, day to day shifts in the patient's internal fluids distribution, and wires being moved. Notably ECG electrodes are generally not electrically shielded. Even textile rubbing over the electrodes can lead to signal distortion. The one or more disturbances could be detected based on a comparison between the test signal portion and the induced ECG test signal, for example by signal processor 16. Since the properties of the ECG test signal induced into the tissue of the patient are completely known, any differences between the induced ECG test signal and the test signal portion of the ECG, as detected during the ECG procedure, can be considered disturbances. Detecting the one or more disturbances in the test signal portion may further include any of the previously discussed techniques of spectral analysis. Step 108 of method 100 includes determining a disturbance level for the ECG test signal. The disturbance level could, for example, be based on a quantity of the disturbances and/or a severity of the disturbances. Optional step 110 of method 100 includes comparing the disturbance level to a disturbance threshold. When the disturbance level exceeds the disturbance threshold, signal processor 16 could automatically reject the ECG heart signals immediately preceding and following the ECG test signal as being of such low quality that they cannot be relied upon to accurately reflect the original signals as generated by the patient's heart. Step 112 of method 100 includes filtering out at least a part of the ECG signal upon detection of the one or more disturbances. This step could include the techniques of spectral analysis previously discussed to isolate and filter out disturbances at a particular frequency. Additionally or alternatively, this step could include rejecting a portion of the signal received at a predetermined time when the severity or number of the disturbances makes filtering impracticable.

Step 114 of method 100 includes determining a fidelity metric for the heart signal portion based on the disturbance level. Since the ECG heart signals and the ECG test signals are detected by the same plurality of electrodes very closely in time, disturbances affecting the ECG test signals will also affect the ECG heart signals. Accordingly, the disturbance level determined for the test signal portion of the ECG signal can also be applied to the heart signal portion. In this way testing how well the detected output of the test signal portion matches the known input of the induced ECG test signals is used as proxy for determining the fidelity of the heart signal portion when the original signals generated by the patient's heart are unknown independent of the ECG measurement. For example, the fidelity metric could be determined based on a morphological comparison between the ECG heart signals and the ECG test signals. In this way, the fidelity metric could include known metrics for quantifying morphological differences between signals, such as the Pearson correlation coefficient, the Spearman correlation coefficient, the dynamic time warping distance, or sample errors between the signal sets (e.g. the root mean square error). In another example, the fidelity metric could be determined based on a frequency comparison between the ECG heart signals and the ECG test signals. In this way, determining the fidelity metric could include calculating a ratio between the two signals (which may further include calculating the ratio just at a band of frequencies of interest) or comparing the signal to noise ratio of the ECG heart signals and ECG test signals at the band of interest. In yet another example, the fidelity metric may be determined based on a time domain comparison between the ECG heart signals and the ECG test signals. In this way, the fidelity metric could include a distance between landmarks within the two sets of signals (e.g. square wave duration or distance between peaks).

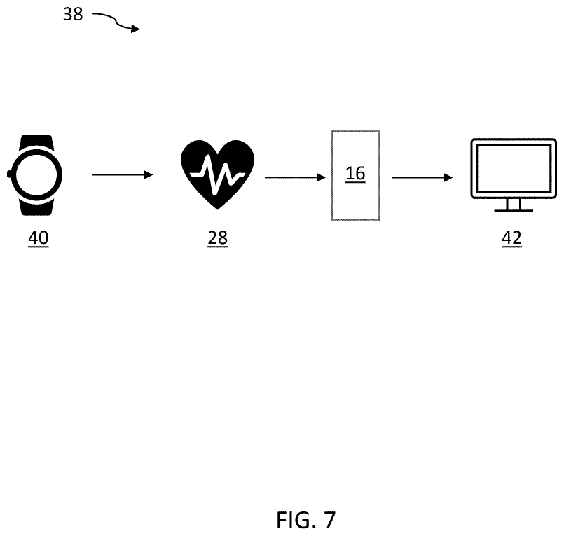

Notably, the systems and methods disclosed herein can improve signal fidelity without filtering out the disturbances. Providing user feedback on the shape, frequency, or amplitude of the disturbances provides helpful information for improving fidelity even without filtering the signals. For example, detecting consistent disturbances at 50 or 60 Hz could indicate to the operator that the ECG set-up needs to be changed to reduce EMI. Feedback can be communicated to a user via a patient monitor, such as display screen 42 shown in FIG. 7. Displaying the artificial ECG test signals along with the ECG hearts signals could confuse a medical trained specialist. To prevent such confusion one can, display the test portion of the ECG signal with a different color. Alternatively, the test signal portion may not be displayed and is only used to correct the ECG signal prior to being displayed. In yet another alternative, the ECG signal with the imposed test signal portion is shown on a separate position of the patient monitor.

Improving ECG signal fidelity may also open-up the use of ECG as a proxy for other biomarkers. For example, ECG with improved fidelity may be used as proxy for glucose levels. Heart rate is a clinical parameter that can be easily derived from ECG measurements. Hypoglycemia has been associated with increased heart rate and alterations to normal heart rate variability. However, the correlation between heart rate and blood glucose levels may depend on time of the day and other factors such as rest, sleep, diet, and activity level. It may well be the case that to use ECG as proxy for glucose one has to consider the time of the day and person's state. This fits well with measuring ECG in a continuous or semi-continuous fashion by, for instance, a wearable device. In addition to being associated with hypoglycemia, in certain circumstances increased heart rate may also be associated with hyperglycemia. As both are detrimental for health, ECG measurement may be used as a proxy for glucose being out of the normal range. A wearable device wrist device can provide a variety of information on a patient's health status which can be combined with a continuous or semi-continuous ECG measurement to create a context for increased heart rate and help indicate if glucose level in blood is increasing or decreasing. Symptoms such as vomiting, excessive hunger, thirst, and/or vision problems can give context to the ECG measurements and if the glucose is high or low. Moreover, besides heart rate, other biomarkers of heart functioning are recorded that are affiliated with heart disorders that can be correlated to glucose level. It is well described that the features of the ECG change during hypoglycemia. These changes include a general slowing of the conduction, as quantified by prolonged QTc and prolonged TpTec. Various other parameters can be derived from the ECG as well. For instance, fibrotic changes, especially in the basal area of the left ventricle, have frequently been observed in diabetic patients (with high glucose level), even when cardiac involvement is clinically not yet evident. The preclinical phase of diabetic cardiomyopathy may be diagnosed by demonstrating exercise-induced left ventricular dysfunction, even when the resting cardiac function is still adequate.

Without the previously discussed systems and methods for determining a fidelity metric of an ECG signal, the disturbance level would be too high to make ECG practicable in this way as a biomarker proxy. This is especially true when the ECG measurements are performed using a wearable consumer device. FIG. 7 illustrates a mobile ECG monitoring system 38. Mobile monitoring system 38 includes a wearable device 40. In some embodiments, the first plurality of electrodes 6 and signal processor 16 are integrated into wearable device 40. Wearable device 40 detects ECG signal 28, including both the heart signal portion and test signal portion. ECG signal 28 is received by signal processor 16, which may be located within wearable device 40 or a separate device. Signal processor 16 determines a fidelity metric for the heart signal portion of ECG signal 28 and displays feedback based on the fidelity metric via a display screen 42. Similar to signal processor 16, display screen 42 may be a screen on wearable device 40 or a separate device.

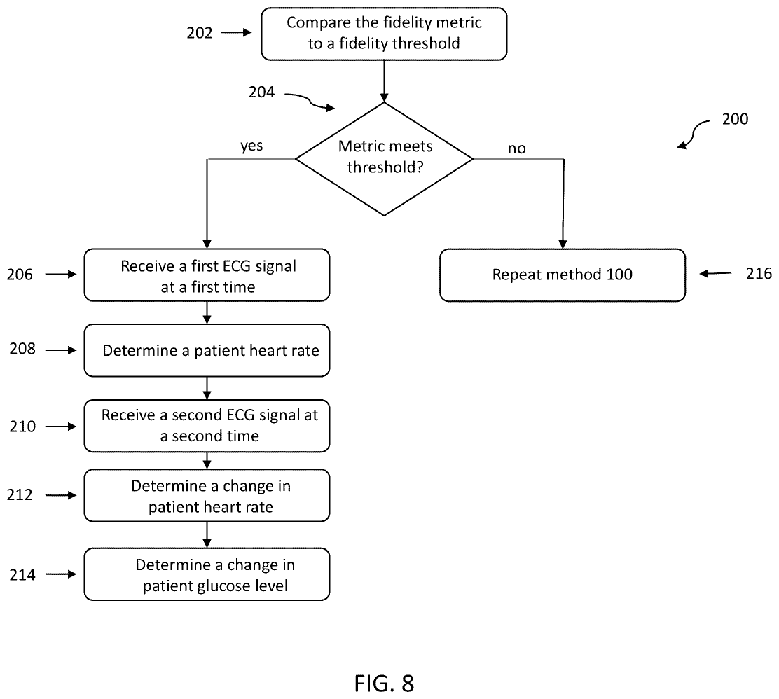

FIG. 8 is a flow chart of a method 200 for determining a change in a patient glucose level according to some aspects of the present disclosure. Step 202 of method 200 includes comparing the fidelity metric to a fidelity threshold. The fidelity metric threshold may be selected according to the particular application. For example, using an ECG signal as a proxy for a biomarker such as blood glucose would typically involve a higher threshold than when using the ECG signal for routine heart health screening. Step 204 of method 100 includes determining whether the fidelity metric meets or exceeds the fidelity threshold. When the fidelity metric fails to meet the fidelity threshold, the ECG signal is deemed to be insufficiently reliable to be used as a proxy for blood glucose. The method proceeds to step 216 and the determination of the fidelity metric is repeated. When the fidelity metric meets or exceeds the fidelity threshold, the method proceeds to step 206 which includes receiving a first ECG signal at a first time. The first ECG signal may be substantially the same as previously discussed ECG signal 28. Step 208 of method 200 includes determining a patient heart rate, based at least partially, on the heart signal portion of the first ECG signal. This can be achieved via any of the well-known methods in the art for determining a heart rate from an ECG measurement. Step 210 of method 200 includes receiving a second ECG signal at a second time. When the patient is being continuously or semi-continuously monitored, for example by wearable device 40, the time between the first ECG signal and second ECG signal may be very brief, for example the time for two heart beats to occur. Step 212 of method 200 includes determining a change in the patient heart rate based, at least partially, on the heart signal portion of the second ECG signal. This could be determined based on a comparison between a heart rate corresponding to the first ECG signal and a heart rate corresponding to the second ECG signal. Step 214 of method 200 includes determining a change in a patient glucose level based, at least partially, on the determined change in heart rate. This step may be accomplished using the previously discussed relationship between elevated heart rate and both hyper and hypoglycemia. This step may be further based on other data indicative of patient health. For example, abnormal motion as detected by a wearable accelerometer may indicate low glucose levels due to exercise.

Another application of the techniques in this disclosure for improving ECG signal fidelity is an improvement in measuring respiratory rate via ECG. A known technique for measuring respiratory rate is measuring the changing impedance of the thoracic cavity during inhalations and exhalations. Returning to FIG. 1, respiration impedance circuit 44 includes an AC signal source applying (driving) a relatively high (but <100 kHz) frequency signal far above the bandwidth of the ECG heart signals 26. The AC circuit signal is generated between the RA and LA electrodes of the second plurality of electrodes 6. A resulting AC voltage is then measured between these electrodes and impedance Z=V_{AC}/I_{AC} is calculated. The impedance Z includes an amplitude modulation component caused by respiration. This small impedance change will result in a voltage (amplitude) change that can detect by filtering the carrier frequency induced onto the body by the AC signal of respiratory impedance circuit 44 and extract the modulated respiration signal on top of this carrier.

Nevertheless, the accuracy and reliability of measuring respiratory rate in this manner requires improvement. All the cables, sensors, electrode skin interfaces etc. in the chain are present in the AM signal. This introduces many opportunities for signal interference. Moreover, when the attachment force is limited and the force on the gel (there is a gel between the human skin and the actual electrode) is slightly changing during measurement, a false signal can occur. The methods and systems for determining a fidelity metric of an ECG signal, as previously discussed, can be used to detect the most stable electrodes, thereby utilizing the best electrode skin interfaces for measurement.

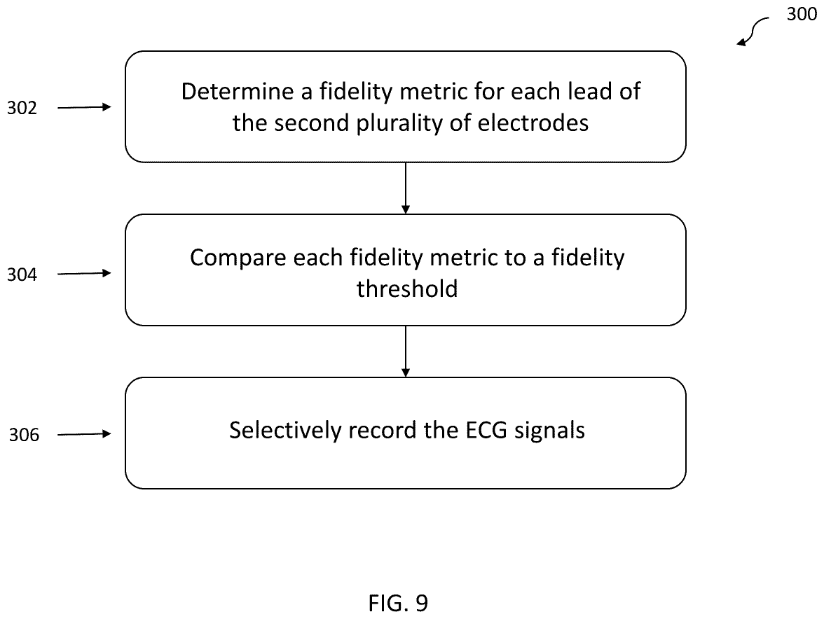

FIG. 9 illustrates a method 300 for selectively recording ECG signals from the second plurality of electrodes 6. Step 302 of method 300 includes determining a fidelity metric for each lead within the second plurality of electrodes. The detected ECG signal 28, including the testing signal portion 24, is received by each lead in the second plurality of electrodes 6. In this way the disturbance of the received signal per lead can be evaluated and will enable classification of the attachment quality (best signal quality due to best skin-electrode interface) of the involved electrode set of the different leads. Moreover, a patient monitor can be used to switch electrodes on and off. In this way each individual electrodes may be tested by testing different combinations of leads. Step 304 of method 300 includes comparing each of the fidelity metrics to a fidelity threshold. This has the advantage of selecting the leads that receive the most optimal signal for deriving respiratory rate, for instance those where the impedance change due to breathing is the largest. Step 304 of method 300 includes selectively recording an ECG signal detected by a lead within the second plurality of electrodes 6 only when the fidelity metric corresponding to the lead meets or exceeds the fidelity threshold. Alternatively, comparing the fidelity metrics to a fidelity metric may not be necessary and the method may selectively record only the lead with the highest fidelity metric.

Accordingly, the systems and methods herein for determining a fidelity metric of an ECG signal not only allow improvements in conventional ECG measurements, but also expand the possible applications of these ECG measurements.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects can be implemented using hardware, software, or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some examples, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific examples described herein. It is, therefore, to be understood that the foregoing examples are presented by way of example only and that, within the scope of the appended claims and equivalents thereto, examples can be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed is:

1. An electrocardiogram (ECG) system, the system comprising:
a signal generator configured to induce an ECG test signal into a tissue of a patient via a first plurality of electrodes;
a second plurality of electrodes configured to attach to skin of the patient to detect an ECG heart signal, and further configured to detect the ECG test signal;
a signal processor configured to:
receive an ECG signal comprising a heart signal portion, based on the detected ECG heart signal, and a test signal portion, based on the detected ECG test signal;
detect one or more disturbances in the test signal portion based on a comparison between the test signal portion and the induced ECG test signal;
determine a disturbance level for the test signal portion based on the one or more disturbances; and
determine a fidelity metric for the heart signal portion based on the disturbance level.

2. The system of claim 1, wherein the signal processor is further configured to filter out at least a portion of the ECG signal upon detection of the one or more disturbances.

3. The system of claim 1, wherein determining the fidelity metric further comprises:
comparing the disturbance level to a disturbance threshold; and
filtering out at least a part of the ECG signal when the disturbance level meets or exceeds the disturbance threshold.

4. The system of claim 1, wherein the signal processor is further configured to selectively control the signal generator so that the ECG test signal shares one or more spectral characteristics with the ECG heart signal.

5. The system of claim 1, wherein the ECG test signal comprises a rectangular pulse wave, and the signal processor is further configured to determine one or more frequencies associated with the one or more disturbances.

6. The system of claim 1, wherein the ECG test signal comprises a sine wave, the signal processor is further configured to selectively control the signal generator to continuously increase the frequency of the sine wave, and the signal processor is further configured to determine one or more frequencies associated with the one or more disturbances based, at least in part, on the changing frequency of the sine wave.

7. The system of claim 1, wherein the first plurality of electrodes and the signal processor are integrated into a wearable device.

8. The system of claim 1, wherein the signal processor is further configured to:
determine a first beat portion and a second beat portion of the ECG heart signal, based at least in part on the heart signal portion; and
selectively control a timing of the signal generator to induce the ECG test signal between the first beat portion and the second beat portion of the ECG heart signal.

9. A method for determining a fidelity metric of an ECG signal, the method comprising:
inducing an ECG test signal into a tissue of a patient via a first plurality of electrodes;
receiving, an ECG signal comprising a heart signal portion and a test signal portion, the heart signal portion being based on an ECG heart signal detected by a second plurality of electrodes, and the test signal portion being based on an ECG test signal also detected with the second plurality of electrodes;
detecting one or more disturbances in the test signal portion based on a comparison between the test signal portion and the induced ECG test signals;
determining a disturbance level for the test signal portion based on the one or more disturbances; and
determining a fidelity metric for the heart signal portion based on the disturbance level.

10. The method of claim 9, further comprising:
receiving a first ECG signal at a first time;
determining a patient heart rate based, at least partially, on the heart signal portion of the first ECG signal;
receiving a second ECG signal at a second time;
determining a change in the patient heart rate based, at least partially, on the heart signal portion of the second ECG signal; and
determining a change in patient glucose level based, at least partially, on the determined change in patient heart rate.

11. The method of claim 9, further comprising:
determining a fidelity metric for each lead within the second plurality of electrodes;
comparing each of the fidelity metrics to a fidelity threshold; and
selectively recording the ECG signal detected by a lead within the second plurality of leads only when the fidelity metric corresponding to the lead meets or exceeds the fidelity threshold.

12. The method of claim 9, further comprising filtering out at least a part of the ECG signal upon detection of the one or more disturbances.

13. The method of claim 9, further comprising:
comparing the disturbance level to a disturbance threshold; and
filtering out at least a part of the ECG signal when the disturbance level exceeds the disturbance threshold.

14. The method of claim 9, further comprising:
selectively controlling a signal generator so that the ECG test signal shares one or more spectral characteristics with the ECG heart signal.

15. The method of claim 9, further comprising
determining a first beat portion and a second beat portion of the ECG heart signal, based at least in part on the heart signal portion; and
selectively controlling a timing of a signal generator to induce the ECG test signal between the first beat portion and the second beat portion of the ECG heart signal.
